(19) United States
(12) Patent Application Publication
(10) Pub. No.: US 2024/0341569 A1
(43) Pub. Date: Oct. 17, 2024

(54) MEDICAL APPARATUS, MEDICIAL SYSTEM AND METHOD FOR MONITORING A SOURCE IMAGE STREAM

(71) Applicant: KARL STORZ SE & Co. KG, Tuttlingen (DE)

(72) Inventor: Klaus BRÜDERLE, Tuttlingen (DE)

(73) Assignee: KARL STORZ SE & Co. KG, Tuttlingen (DE)

(21) Appl. No.: 18/635,837

(22) Filed: Apr. 15, 2024

(30) Foreign Application Priority Data
Apr. 17, 2023 (DE) 10 2023 109 621.4

(51) Int. Cl. A61B 1/00; G06T 7/00

(52) U.S. Cl. CPC A61B 1/000096; A61B 1/00045; A61B 1/00055; G06T 7/0012; G06T 2207/10068

(57) ABSTRACT

A medical apparatus for monitoring a source image stream with a plurality of successive source images, the apparatus comprising: a processing device designed to receive the source image stream and superimpose a line of a defined test pattern on the source images of the source image stream, with the test pattern rotating continuously according to a defined specification over the temporal progression of the source images, and to transmit the source images, superimposed thus, as transmission images of a transmission image stream; and a verification device designed to receive the transmission image stream, check a plurality of conditions in relation to the transmission images of the transmission image stream, and output a warning signal if at least one condition is not met. A corresponding medical system and a method are also disclosed.

### CROSS-REFERENCE TO RELATED APPLICATIONS

This application claims priority of German Patent Application No. 10 2023 109 621.4 filed on Apr. 17, 2023, the contents of which are incorporated herein.

### TECHNICAL FIELD

The description relates to a medical apparatus, a medical system and a method for monitoring a source image stream with a plurality of successive source images.

### BACKGROUND

The most important requirement in the field of medical video endoscopy is that of providing users with an image stream from the endoscope camera for observation purposes. For example, this occurs within the scope of an examination or operation by virtue of the motif scenes being presented live or being recorded. Additionally, accompanying image, media or data streams are frequently also processed.

Within the scope of current technology, checking an image stream predominantly relates to the development procedure. In this case, the plausibility of an image visualization or recording is usually implemented in human-physiological fashion. Specifically, a tester visually checks an image stream for currentness, completeness and presence of artifacts over an appropriate period of time.

Sometimes, a test image is also fed in and evaluated at the target within the scope of an automated test. For example, green and red images are transmitted alternately and their reception is checked. This is implemented in a dedicated test environment. For worst-case or operational-life statements, this dedicated test network is modified in respect of its capability or performance. A reliability prediction regarding all our system combinations is then made on the basis of these results from the test environment. There is no verification during the operational life to date.

It is therefore an object to disclose an improved medical apparatus, an improved medical system and an improved method for monitoring a source image stream with a plurality of successive source images, which also enable continuous monitoring during the operational life.

### SUMMARY

According to a first aspect, this object is achieved by a medical apparatus for monitoring a source image stream having a plurality of successive source images, with the apparatus comprising: a processing device designed to receive the source image stream and superimpose a line of a defined test pattern on the source images of the source image stream, with the test pattern rotating continuously according to a defined specification over the temporal progression of the source images, and to transmit the source images, superimposed thus, as transmission images of a transmission image stream; a verification device designed to receive the transmission image stream, check a plurality of conditions in relation to the transmission images of the transmission image stream, and output a warning signal if at least one condition is not met, with a first condition being that the defined test pattern could be identified at least in a defined percentage of the transmission images and a second condition being that a rotation of the test pattern according to the defined specification could be identified in these transmission images. This apparatus enables continuous checking as to whether the transmission images are received in the correct order on an ongoing basis and also contain ongoing image information, i.e. these images are not static on account of an error or contain static image regions which should actually exhibit changes even though images are received in the correct order on an ongoing basis.

For example, the test pattern can be chosen as a polygon, in particular as a triangle, quadrilateral or star. A rounded shape, in particular an oval, can also serve as a test pattern. It is even possible to use a circle, with the latter then rotating not about its center but in off-centered fashion. Moreover, the line of the test pattern can be interrupted at at least one point in order to identify the orientation of the test pattern more easily or more reliably. The test pattern can be integrated dynamically into the source images, i.e. the original motif. Moreover, the test pattern can also be created easily.

Moreover, however, the test pattern is also detectable and retrievable in a comparatively simple manner from a mathematical point of view. This is also true if the source images are subsequently subjected to changes, for example a change in size, a modification of the aspect ratio, when image parts are cut out, separated or merged, or overlaid with further information.

The test pattern is chosen such that it interacts, specifically sweeps over, each part of the image or at least each region of interest (ROI) in the image over time. Finally, the test pattern is not only easily detectable by means of image processing mathematics but also easy to process or visualize by the human eye. It may also be advantageous for the test pattern to have exactly one start point and exactly one end point.

The present technical solution also offers advantages in the case of a plurality of media stream contents, for example image data streams, which are brought to a user for a live view or for composed recording. In this case, it is desirable for the image streams to be displayed not only in up-to-date fashion and in the correct order, but also synchronously with one another.

Moreover, the data stream, specifically the source image data stream, begins to be monitored not only once the camera or the imager has supplied the source image but already at the point of motif capture—i.e. in front of or at least in the imager itself. In particular, the transmission image stream is transmitted via an equipment cable and/or a network, wirelessly or in a wired fashion.

The defined shape of the test pattern allows various checks to be carried out if the images or image segments are separated, segmented and/or recombined. The courses of the segment boundaries can be checked at the boundaries of image segments. The image segments are positioned correctly if the line of the test pattern extends continuously, i.e. without offsets or discontinuities.

In so doing, aspect ratios can also be checked or adapted at the same time. If the line of the test pattern runs continuously, then it is also very likely that the aspect ratios of a plurality of segments fit to one another.

Thus, proposed here are the interaction of the original motif and the introduction of a test pattern into the source image. To the greatest extent possible, this introduction should be implemented at the origin of the image stream. However, it may also be expedient to only implement this at another point in the image stream path, with however checking then only being possible from this point onward. The evaluation or verification should take place at the end point to the greatest extent possible. In principle, this may also occur earlier, but then the subsequent part of the path cannot be checked.

Should the test pattern be introduced so as to be visible, it is proposed that this introduction of the test pattern is only active at the setup or switch-on time for an evaluation during the operational life. This allows a check to be carried out before the actual use, within the scope of a referencing procedure so to speak. In an alternative or in addition, it is possible to perform such a check after the actual use has finished, in particular for verification or documentation purposes.

The procedure disclosed here offers an apparatus for a general testing method and a testing method as such for all types of image stream transport. In so doing, it can preferably be used as a testing method for development and operational life environments. In this case, the testing method can be implemented in fully automated fashion, including the verification and an identification of errors. In this context, the documentation can be performed once, in particular as individual evidence, or repeatedly, in particular as a proof of quality.

This completely achieves the object.

In a preferred configuration, the test pattern has a spiral shape.

A further advantageous evaluation is possible on account of the mathematically unique description of the spiral shape. Let the assumption be made that all that is available at a later time is an image portion of a transmission image containing one or more sections of the spiral shape. These sections can be referred to as spiral line segments. Conversely, the mathematical description of the spiral shape enables the use of one of these spiral line segments to ascertain the center of the transmission image to which the image portion belongs.

For the check, a straight line could be drawn through the origin of the spiral shape in the transmission image, the points of intersection with the spiral could be identified and a mathematical test could be carried out with regards to whether these are plausible, i.e. whether these points of intersection correspond to the defined spiral shape.

Moreover, the spiral shape assists a two-factor calculation and remains substantially uninfluenced with regards to the conventional image transport or image compression methods, and so the line in the spiral shape remains identifiable even over relatively protracted processing and transfer processes, for example also including a compression (e.g. as h264 or MJPEG).

In a preferred configuration, the spiral shape is an Archimedean spiral.

The Archimedean spiral can be described using the formulas x=k*phi*cos(phi) and y=k*phi*sin(phi) in the Cartesian coordinate system, where phi can be understood to be an angle and k is a stretching or compression factor for the spiral. Conversely, phi=a tan(y/x) also applies in that case. The Archimedean spiral has the property that any straight line running through its origin intersects the spiral multiple times, with these points of intersection being spaced apart equidistantly along the straight line. Since the stretching or compression factor k is the same for the x-and the y-coordinate in this case, it is also possible to check the condition that the spiral shape is in fact circular and not shaped elliptically. If an elliptic spiral shape is discovered in place of a circular spiral shape, then this would suggest a compression or stretching of the transmission image.

In a preferred configuration, the line is embodied in a contrast color which cannot be found, or can only be found to a statistically negligible extent, in the source images.

This configuration makes it particularly easy to find the spiral shape line for the check. In particular, the contrast colors include white (RGB 255,255,255) and black (RGB 0,0,0).

In a preferred configuration, an origin of the spiral shape is located in the center of the source images.

This configuration allows checking of a further condition that the spiral must end at the image center.

In a preferred configuration, a piece of backup image information is stored for each transmission image of the transmission image stream, the backup image information containing the image information from the respective source image that is overlaid by the line of the test pattern.

This configuration ensures that the image information that was overlaid by the test pattern is not lost. In this case, the backup image information is preferably positioned in an edge region of the transmission image or transmitted as meta-information together with the respective transmission image. In this context, the backup image information can specifically contain the pixel information along the spiral shape or be designed as a mask.

In a preferred configuration, the apparatus also comprises a restoration apparatus, which is designed to restore the line of the test pattern in the transmission image using the associated backup image information and to output the transmission images, restored thus, as presentation images of a presentation image stream.

This configuration reduces or removes the perceptibility of the superimposed test pattern for the user. If the backup image information was stored as a mask, then the respective transition image can easily be combined with its respective backup image information in order to form a presentation image. In particular, this can be implemented after a transmission image has been checked, but also in parallel with this check.

In a preferred configuration, the test pattern additionally has a straight path which leads radially outward from an origin or center of the test pattern.

In this configuration, the straight path visualizes the progress in the image stream particularly clearly in the style of a pointer. A uniformly progressing rotation of the pointer indicates a correct sequence and speed of the transmission images. If a plurality of image streams are intended to be checked with regards to mutual synchronicity, then it is possible to simply compare the pointers in the image streams with one another in respect of their angle. If a deviation is determined, synchronicity can be reestablished by deceleration, acceleration or jumps in one of the image streams.

In a preferred configuration, the defined specification contains the spiral rotating inwardly.

As mentioned previously, the spiral rotates during a verification interval. A certain degree of image dynamics is provided by way of this rotation. The spiral rotating inwardly, i.e. clockwise, is considered suitable.

In a preferred configuration, the plurality of conditions include at least one further condition: the line of the test pattern is fully identifiable in the transmission image in a defined further percentage.

In this configuration, it is possible to check whether compression losses potentially deteriorate the image quality.

In a preferred configuration, the apparatus also comprises an output apparatus, which is designed to output the warning signal so that the latter is perceptible in visual, acoustic and/or haptic fashion.

In this configuration, the user is notified when the actual transmission image stream deviates from the expected target transmission image stream. In that case, the user is aware that an actual transmission image stream might not show what the user would expect in the case of an ideal, error-free transmission. The warning signal may also contain information about the type of warning, which is to say about the type and severity, with regards to the condition or conditions that have not been met. In that case, it is possible that the user can select whether and how they wish to be notified with regards to the respective deviations from the desired target transmission image stream.

In a preferred configuration, the processing device is further designed to invert the transmission images during defined intervals.

This configuration contains further image dynamics that can be checked in the transmission images for the purpose of monitoring the transmission image stream. If the verification device cannot identify such changes between activated inversion and deactivated inversion or cannot identify such changes at a spacing of the defined intervals, then this can be signaled as a potential error situation.

According to a second aspect, the object is achieved by a medical system for monitoring an image stream with a plurality of successive images, comprising an imager for creating the source image stream, an apparatus as claimed in any of the preceding claims, and a display apparatus designed to output the transmission image stream and reproduce the warning signal, with the processing device being integrated in the imager in particular.

According to a second aspect, the object is achieved by a method for monitoring a source image stream with a plurality of successive source images, the method including the following steps: receiving the source image stream; superimposing a line of a defined test pattern on the source images of the source image stream, with the test pattern rotating continuously according to a defined specification over the temporal progression of the source images; transmitting the source images, superimposed thus, as transmission images of a transmission image stream; receiving the transmission image stream; checking a plurality of conditions in relation to the transmission images of the transmission image stream; and outputting a warning signal if at least one condition is not met, with a first condition being that the defined test pattern could be identified at least in a defined percentage of the transmission images and a second condition being that a rotation of the test pattern according to the defined specification could be identified in these transmission images.

It will be appreciated that the features mentioned above and the features yet to be explained below are applicable not only in the combination specified in each case but also in other combinations or on their own, without departing from the scope of the present description.

### BRIEF DESCRIPTION OF THE DRAWINGS

Exemplary embodiments of the description are depicted in the drawing and are described in more detail in the following description.

FIG. 1 shows an embodiment of a medical system for monitoring a source image stream with a plurality of successive source images, having an embodiment of a medical apparatus for monitoring a source image stream with a plurality of successive source images;

FIG. 2 shows an embodiment of a method for monitoring a source image stream with a plurality of successive source images;

FIG. 3 shows an embodiment of a source image;

FIG. 4 shows an embodiment of a test pattern as a spiral shape;

FIG. 5 shows an embodiment of a transmission image which arises from the superposition of the source image from FIG. 3 and the spiral shape from FIG. 4.

FIG. 6 shows an embodiment of a transmission image, in which the spiral shape has rotated proceeding from FIG. 5;

FIG. 7 shows an embodiment of a transmission image, in which the spiral shape has rotated further proceeding from FIG. 5; and

FIG. 8 shows an embodiment of a transmission image, in which the spiral shape has rotated further once again proceeding from FIG. 5.

### DETAILED DESCRIPTION OF PREFERRED EMBODIMENTS

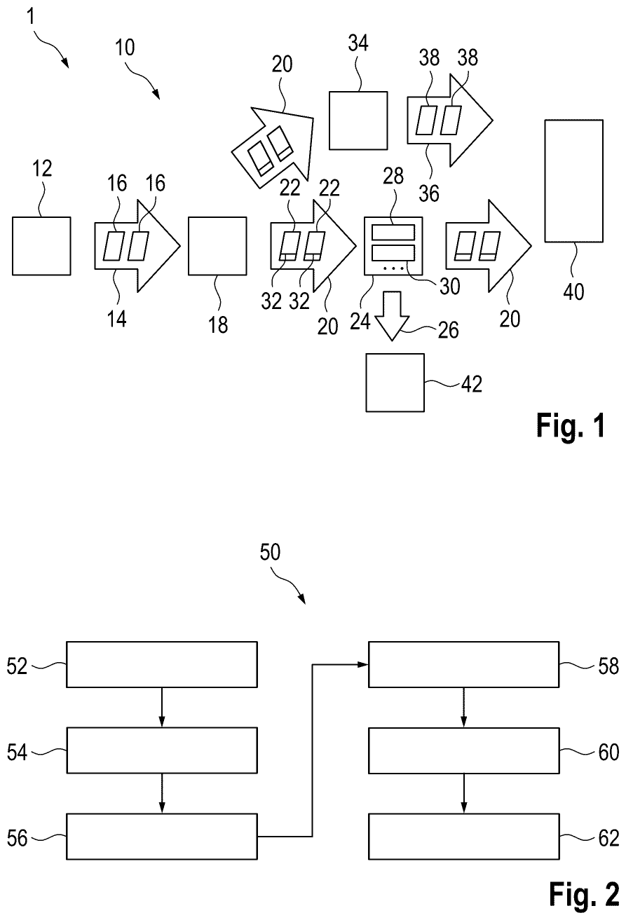

FIG. 1 shows a medical system 1 for monitoring a source image stream 14 with a plurality of successive source images 16, comprising an imager 12 for creating the source image stream 14, a medical apparatus 10 for monitoring a source image stream 14 with a plurality of successive source images 16, and a display apparatus 40 which is designed to output the transmission image stream and reproduce the warning signal.

The apparatus 10 comprises various elements, including a processing device 18 designed to receive the source image stream 14 and superimpose a line 70 (see FIG. 4) of a defined test pattern on the source images 16 of the source image stream 14, with the test pattern rotating continuously according to a defined specification over the temporal progression of the source images 16, and to transmit the source images 16, superimposed thus, as transmission images 22 of a transmission image stream 20. In particular, the processing device 18 is integrated in the imager 12. In this case, the test pattern is a defined spiral shape.

The apparatus 10 also comprises a verification device 24 designed to receive the transmission image stream 20, check a plurality of conditions 28, 30 in relation to the transmission images 22 of the transmission image stream 20, and output a warning signal 26 if at least one condition 28, 30 is not met.

The plurality of conditions 28, 30 include a first condition 28 that the defined spiral shape could be identified at least in a defined percentage of the transmission images 22, and a second condition 30 is that a rotation of the spiral shape according to the defined specification could be identified in these transmission images 22.

In this embodiment, a piece of backup image information 32 is stored for each transmission image 22 of the transmission image stream 20, the backup image information containing the image information from the respective source image 16 that is overlaid by the line 70 of the spiral shape.

The backup image information 32 is used by a restoration apparatus 34, which is designed to restore the line 70 of the spiral shape in the transmission image 22 using the associated backup image information 32 and to output the transmission images 22, restored thus, as presentation images 38 of a presentation image stream 36.

The apparatus 10 also comprises an output apparatus 42, which is designed to output the warning signal 26 so that the latter is perceptible in visual, acoustic and/or haptic fashion. Moreover, the processing device 18 of this embodiment is further designed to invert the transmission images 22 during defined intervals.

FIG. 2 shows a method 50 for monitoring a source image stream 14 with a plurality of successive source images 16. The embodiment of the shown method 50 starts with the reception 52 of the source image stream 14, followed by the source images 14 of the source image stream 16 being overlaid 54 by a line 70 in a defined spiral shape, with the spiral shape rotating continuously according to a defined specification over the temporal progression of the source images 16.

The method 50 continues with the transmission 56 of the source images 16, overlaid thus, as transmission images 22 of the transmission image stream 20, the reception 58 of the transmission image stream 20 and the checking 60 of a plurality of conditions 28, 30 in relation to the transmission images 22 of the transmission image stream 20. A warning signal 26 is output 62 if at least one condition 28, 30 is not met.

The following also holds true here again: A first condition 28 is that the defined spiral shape could be identified at least in a defined percentage of the transmission images, and a second condition 30 is that a rotation of the spiral shape according to the defined specification could be identified in these transmission images 22.

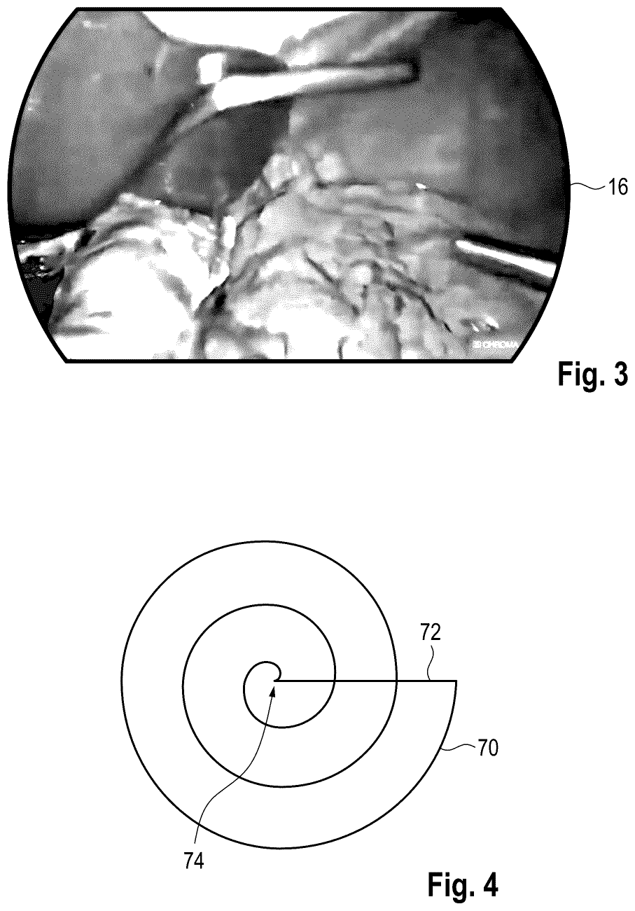

FIG. 3 shows an embodiment of a source image 16.

FIG. 4 shows an embodiment of a test pattern, in this case a spiral shape, as a line 70. In this case, the spiral shape is an Archimedean spiral. The line 70 is embodied in a contrast color, black in this case, which cannot be found or can only be found to a statistically negligible extent, in the source images 16. The spiral shape additionally has a straight path 72 which leads radially outward from an origin 74 of the spiral shape.

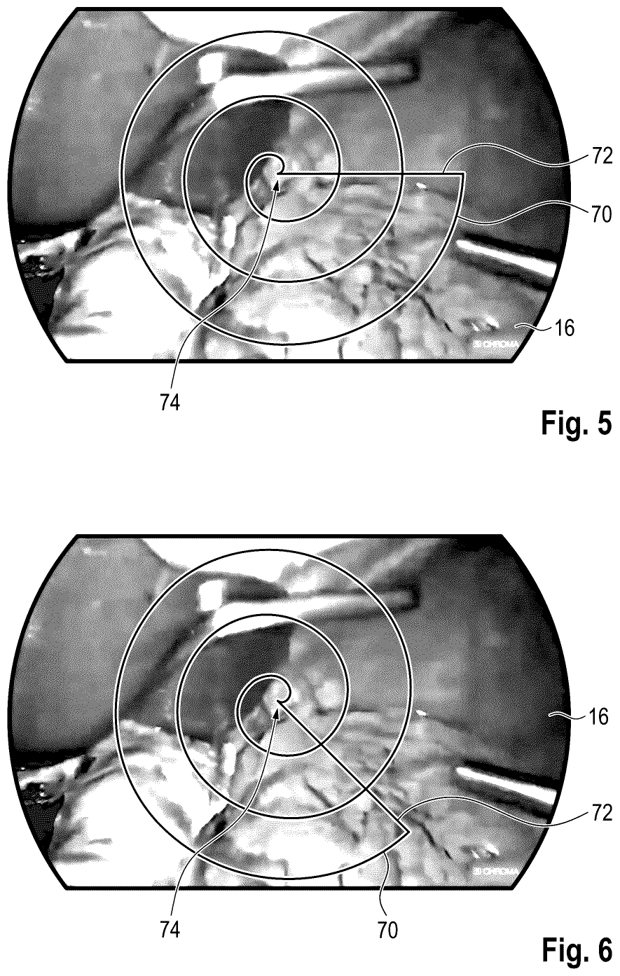

FIG. 5 shows an embodiment of a transmission image 22 which arises from the superposition of the source image 16 from FIG. 3 and the line 70 in spiral shape from FIG. 4.

FIG. 6 shows an embodiment of a transmission image 22, in which the spiral shape has rotated proceeding from FIG. 5.

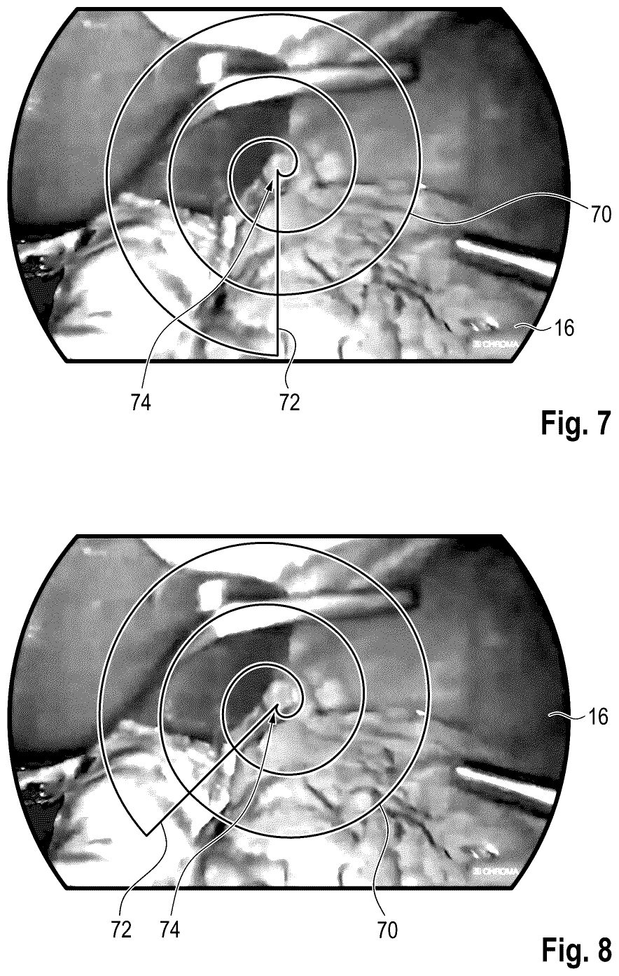

FIG. 7 shows an embodiment of a transmission image 22, in which the spiral shape has rotated further proceeding from FIG. 5; and

FIG. 8 shows an embodiment of a transmission image 22, in which the spiral shape has rotated further once again proceeding from FIG. 5.

1. A medical apparatus for monitoring a source image stream with a plurality of successive source images, the apparatus comprising:
a processing device designed to receive the source image stream and superimpose a line of a defined test pattern on the source images of the source image stream, with the test pattern rotating continuously according to a defined specification over the temporal progression of the source images, and to transmit the source images having the superimposed line, as transmission images of a transmission image stream;
a verification device designed to receive the transmission image stream, check a plurality of conditions in relation to the transmission images of the transmission image stream, and output a warning signal if at least one condition is not met,
with a first condition being that the defined test pattern could be identified at least in a defined percentage of the transmission images and a second condition being that a rotation of the test pattern according to the defined specification could be identified in these transmission images.

2. The apparatus as set forth in claim 1, wherein the test pattern has a spiral shape.

3. The apparatus as set forth in claim 2, wherein the spiral shape is an Archimedean spiral.

4. The apparatus as set forth in claim 1, wherein the line is embodied in a contrast color which cannot be found, or can only be found to a statistically negligible extent, in the source images.

5. The apparatus as set forth in claim 2, wherein an origin of the spiral shape is located in the center of the source images.

6. The apparatus as set forth in claim 1, wherein a piece of backup image information is stored for each transmission image of the transmission image stream, the backup image information containing the image information from the respective source image that was overlaid by the line of the test pattern.

7. The apparatus as set forth in claim 6, further having a restoration apparatus, which is designed to restore the line of the test pattern in the transmission image using the associated backup image information and to output the transmission images, restored thus, as presentation images of a presentation image stream.

8. The apparatus as set forth in claim 1, wherein the test pattern additionally has a straight path which leads radially outward from an origin or center of the test pattern.

9. The apparatus as set forth in claim 2, wherein the defined specification contains the spiral rotating inwardly.

10. The apparatus as set forth in claim 1, wherein the plurality of conditions include at least one further condition: the line of the test pattern is fully identifiable in the transmission image in a defined further percentage.

11. The apparatus as set forth in claim 1, further having an output apparatus, which is designed to output the warning signal so that the latter is perceptible in visual, acoustic and/or haptic fashion.

12. The apparatus as set forth in claim 1, wherein the processing device is further designed to invert the transmission images during defined intervals.

13. A medical system for monitoring a source image stream with a plurality of successive source images, comprising:
an imager for creating the source image stream;
an apparatus as set forth in claim 1; and
a display apparatus designed to output the transmission image stream and reproduce the warning signal, with the processing device being integrated in the imager.

14. A method for monitoring a source image stream with a plurality of successive source images, the method including the following steps:
receiving the source image stream;
superimposing a line of a defined test pattern on the source images of the source image stream, with the test pattern rotating continuously according to a defined specification over the temporal progression of the source images;
transmitting the source images, superimposed thus, as transmission images of a transmission image stream;
receiving the transmission image stream;
checking a plurality of conditions in relation to the transmission images of the transmission image stream; and
outputting a warning signal if at least one condition is not met,
with a first condition being that the defined test pattern could be identified at least in a defined percentage of the transmission images and a second condition being that a rotation of the test pattern according to the defined specification could be identified in these transmission images.

15. The method as claimed in claim 14, wherein the test pattern has a spiral shape which is an Archimedean spiral in particular.
